(19) United States
(12) Patent Application Publication
(10) Pub. No.: US 2011/0257414 A1
(43) Pub. Date: Oct. 20, 2011

(54) CATALYST MOLDED BODIES AND METHOD FOR PRODUCING MALEIC ACID ANHYDRIDE

(75) Inventors: Cornelia Katharina Dobner, Ludwigshafen (DE); Stefan Altwasser, Wachenheim (DE); Hagen Wilmer, Ludwigshafen (DE); Frank Rosowski, Mannheim (DE)

(73) Assignee: BASF SE, Ludwigshafen (DE)

(21) Appl. No.: 13/141,179

(22) PCT Filed: Dec. 21, 2009
(86) PCT No.: PCT/EP2009/067657

§ 371 Date: Jun. 21, 2011

(30) Foreign Application Priority Data
Dec. 22, 2008 (EP) 08172633.3

(51) Int. Cl. C07D 307/60; B01J 37/08; B01J 37/04; B01J 27/198; B01J 35/10

(52) U.S. Cl. 549/256; 502/209

(57) ABSTRACT

The catalytically active mass of a catalyst molded body comprises a multi-element oxide containing vanadium and phosphorus. The specific pore volume PV (in ml/g) of the catalyst molded body, the bulk density p of the catalyst molded body (in kg/l), the geometric surface area A_{geo} (in mm²), and the geometric volume V_{geo} (in mm³) of the catalyst molded body satisfy the condition 0.275<PV·ρ·A_{geo}/V_{geo}. In a method for producing maleic acid anhydride by heterogeneously catalytic gas phase oxidation of a hydrocarbon, the catalyst molded body allows a lower pressure loss and a high yield.

The present invention relates to a shaped catalyst body and a process for preparing maleic anhydride by heterogeneously catalyzed gas-phase oxidation and a process for producing the catalyst.

Maleic anhydride is an important intermediate in the synthesis of γ-butyrolactone, tetrahydrofuran and 1,4-butanediol, which are in turn used as solvents or are, for example, processed further to give polymers such as polytetrahydrofuran or polyvinylpyrrolidone.

The preparation of maleic anhydride by oxidation of hydrocarbons such as n-butane, n-butenes or benzene over suitable catalysts has been known for a long time. In general, vanadium-, phosphorus- and oxygen-comprising catalysts, in particular catalysts based on vanadyl pyrophosphate (VO)₂P₂O₇ (known as VPO catalysts), are used for this purpose.

Since the oxidation of the abovementioned hydrocarbons to maleic anhydride is strongly exothermic, the reaction is generally carried out in a shell-and-tube reactor cooled by means of a salt bath. Depending on the size of the plant, this reactor has from a few thousand to several tens of thousands of catalyst-filled tubes. The heat of reaction evolved is transferred via the wall of the catalyst-filled tubes to the surrounding salt bath, generally a eutectic mixture of potassium and sodium nitrate and nitrite, and removed thereby. The individual tubes have a relatively small cross section so that the heat of reaction is removed uniformly and precise maintenance of the temperature can be ensured over the tube cross section. In addition, the reactors are ideally slim and tall, so that the thermal stresses can be absorbed by the tube plates.

When a gas flows through a bed of bulk material, a pressure drop occurs due to friction of the gas stream against the particles. The pressure drop determines the pressure gradient which has to be overcome between the reactor inlet and the reactor outlet. The thin and long configuration of the reactor tubes necessarily results in a comparatively high pressure drop. However, excessively high pressure drops are disadvantageous; they lead to a higher compressor power being required, as a result of which capital and operating costs for the plant are increased and in the case of a limited available compressor power, the productivity of the reactor is reduced.

One possible way of reducing the pressure drop is the use of low-pressure-drop geometries of the shaped catalyst bodies. The shaped body geometry determines the resistance experienced by the gas flowing through the catalyst particles. Relatively large catalyst particles generally give a lower pressure drop, but the external surface area and thus the activity of the catalyst decrease at the same time, so that yield and productivity are reduced.

Increasing the microstructure of heterogeneous catalysts by concomitant use of pore formers is known. U.S. Pat. No. 5,275,996 describes a porous phosphorus/vanadium oxide catalyst for producing a carboxylic anhydride. At least 5% of the pore volume of the catalyst is formed by pores having a diameter of at least 0.8 μm and at least 4% of the pore volume of the catalyst is formed by pores having a diameter of at least 10 μm. In the production of the catalyst, from 4 to 16% of pore formers are mixed with a particulate phosphorus/vanadium oxide catalyst precursor.

Improving the shaped body geometry is subject matter of a number of patent publications. U.S. Pat. No. 4,283,307 discloses an oxidation catalyst in the form of a pellet having a central hole.

U.S. Pat. No. 5,168,090 describes shaped catalyst bodies whose external surface has at least one hollow space and whose geometric volume corresponds to from 30 to 67% of the volume of the hollow-space-free geometric shape and which have a ratio of the external geometric surface area to the geometric volume of at least 20 cm⁻¹. Specifically, U.S. Pat. No. 5,168,090 discloses cylinders having 3 equidistant grooves in the external surface which run parallel to the axis of the cylinder.

WO 01/68245 discloses a catalyst for preparing maleic anhydride by heterogeneously catalyzed gas-phase oxidation, which catalyst has an essentially hollow cylindrical structure which has a particular ratio of the height to the diameter of the through-hole and a particular ratio of the geometric surface area to the geometric volume.

WO 03/078057 describes a catalyst for preparing maleic anhydride, which catalyst comprises a catalytically active composition comprising vanadium, phosphorus and oxygen and has an essentially hollow cylindrical structure and a geometric density dₚ which satisfies particular conditions.

WO 2007/051 602 describes shaped catalyst bodies for preparing maleic anhydride, where the geometric base body enveloping the shaped catalyst body is a prism and the shaped catalyst body is provided with three through-holes. The shaped catalyst body should have a triangular cross section with rounded corners.

It is an object of the present invention to provide a process for preparing maleic anhydride by heterogeneously catalyzed gas-phase oxidation of a hydrocarbon, which combines a low pressure drop with a high yield.

According to the invention, the object is achieved by a shaped catalyst body whose catalytically active composition comprises a vanadium- and phosphorus-comprising multielement oxide, wherein the specific pore volume PV (in ml/g) of the shaped catalyst body, the bulk density ρ of the shaped catalyst body (in kg/l), the geometric surface area A_{geo} (in mm²) and the geometric volume V_{geo} (in mm³) of the shaped catalyst body fulfill the condition:

0.275<PV·ρ·A_{geo}/V_{geo}.

In preferred embodiments

0.30<PV·ρ·A_{geo}/V_{geo}.

For the purposes of the present patent application, the bulk density ρ is the bulk density of the shaped catalyst body in a tube having a circular cross section and an internal diameter of 21 mm. The bulk density of the shaped catalyst body depends on the size and shape of the cross section of the reaction tube because the packing density of the material is lower at the walls (edge effect). Conventional reaction tubes generally have a diameter of from 20 to 25 mm. The bulk density ρ is advantageously determined by filling a model tube having a known volume with shaped catalyst bodies and determining the weight of the shaped catalyst bodies. The bulk density determined here by means of a model tube is a sufficient approximation to the bulk density of the shaped catalyst body in conventional reaction tubes.

The bulk density ρ of the shaped catalyst body in the reaction tube influences the pressure drop observed, with the pressure drop generally increasing with increasing bulk density. In preferred embodiments, the bulk density ρ is less than 0.60 kg/l, preferably less than 0.55 kg/l, in particular less than 0.50 kg/l, e.g. from 0.40 to 0.50 kg/l.

The specific pore volume PV is the (integrated) specific pore volume determined by mercury porosimetry in accordance with DIN 66133. For most solids, mercury behaves as a nonwetting liquid. Mercury is therefore not spontaneously absorbed by the porous material but penetrates into the pores of the sample of solid only under an external pressure. The magnitude of this pressure depends on the size of the pores. This behavior is exploited in Hg porosimetry to measure the pore radius via the volumetric intrusion under an externally applied pressure.

In preferred embodiments, the specific pore volume PV is at least 0.30 ml/g, preferably at least 0.35 ml/g, e.g. from 0.38 to 0.50 ml/g.

Preference is given to at least 15% of the specific pore volume being formed by pores having a size of from 0.3 to 20 μm. It has been found that shaped catalyst bodies having a high proportion of pores in this size range lead to an increase in activity. These pores presumably act as transport pores.

The geometric shape of the shaped catalyst bodies is not subject to any particular restrictions. The bodies can be prisms, cylinders or have other shaped body geometries which can be produced readily, e.g. by extrusion or tabletting, and offer satisfactory mechanical stability.

The ratio of the geometric surface area A_{geo} to the geometric volume V_{geo} is preferably at least 1.50 mm⁻¹, e.g. from 1.50 to 2.60 mm⁻¹, more preferably at least 1.60 mm⁻¹, in particular at least 1.85 mm⁻¹. The geometric volume and the geometric surface area can be calculated from the corresponding measured values of the perfect parent geometric shapes. For example, the geometric volume and the geometric surface area of a hollow cylinder can be calculated on the basis of the height h of the cylinder, the external diameter d₁ and the diameter of the internal hole d₂. The geometric surface area A_{geo} is an idealized parameter and does not take into account the enlargement in the surface area due to the porosity or surface roughness of the shaped bodies.

The ratio A_{geo}/V_{geo} can be increased by providing hollow spaces or recesses on the outer surfaces of the shaped body or holes through the shaped body. The recesses can, for example, be grooves which run parallel to the longitudinal axis or run helically in the cylindrical wall of a cylinder.

Shaped catalyst bodies having an essentially cylindrical body having a longitudinal axis, where the cylindrical body has at least one internal hole, e.g. from one to four internal holes, which run(s) right through the body essentially parallel to the cylindrical axis of the body have been found to be particularly useful. Particularly preferred shaped catalyst bodies have one or four internal hole(s). The term “essentially” indicates that deviations from the ideal geometry, for example slight deformations in the circular structure, end faces which are not flat and parallel, chipped corners and edges, surface roughness or notches in the cylindrical surface, the end faces or the internal surfaces of the through-holes, are encompassed in the case of the shaped catalyst body of the invention. The internal holes preferably have a round or oval cross section, in particular a round cross section. In general, all internal holes have the same cross section.

If the shaped body has more than one internal hole, the central axes of the internal holes are preferably located equidistantly on a cylindrical surface which is concentric with the surface of the cylindrical body. The ratio of the diameter d₂ of an internal hole to the external diameter d₁ of the cylindrical body is preferably from 0.2 to 0.35. The ratio of the diameter d₃ of the cylindrical surface on which the central axes of the internal holes are located to the external diameter d₁ of the cylindrical body is preferably 0.8 to 0.9. To obtain satisfactory mechanical stability, preference is given to both the smallest distance between the internal holes and the smallest distance between the internal holes and the outer cylindrical surface of the body being in each case at least 7% of the diameter d₁ of the cylindrical body.

The ratio of the height h of the cylindrical body to the diameter d₂ of the internal holes is preferably not more than 3.4, in particular from 2.0 to 2.35.

The lateral compressive strength of the shaped catalyst bodies is preferably at least 8 N, in particular at least 10 N. When the shaped body is not rotationally symmetrical and the lateral compressive strength depends on the orientation of the shaped body relative to the force applied, the lateral compressive strength is taken to be the smallest lateral compressive strength.

The invention also provides a process for producing a shaped catalyst body, in which a vanadium- and phosphorus-comprising multielement oxide or a precursor thereof (hereinafter also referred to as catalyst precursor or precursor powder) is mixed with a pore former, the mixture is shaped to give shaped bodies and the shaped bodies are calcined.

Preference is given to using from 18 to 40% by weight, in particular from 20 to 25% by weight, of pore former, based on the weight of the multielement oxide or precursor thereof.

The pore former is preferably particulate and has, in particular, a particle size distribution having an average particle diameter d₅₀ in the range from 1 to 80 μm. The particle size distribution is advantageously determined by means of a laser light scattering instrument Malvern Mastersizer S from Malvern Instruments and a dry measuring dispersing system RODOS from Sympatec.

The multielement oxide or the precursor thereof preferably has a particle size distribution having an average particle diameter d₅₀ in the range from 50 to 70 μm. The average particle size distribution can advantageously be determined in suspension in isobutanol by laser light scattering (Malvern Mastersizer S with a wet dispersing unit MS1).

The atomic ratio of phosphorus/vanadium in the catalytically active composition of the catalyst is generally from 0.9 to 1.5, preferably from 0.9 to 1.2, in particular from 1.0 to 1.1. The average oxidation state of the vanadium is preferably from +3.9 to +4.4 and preferably from 4.0 to 4.3. Suitable active compositions are described, for example, in the patent documents U.S. Pat. No. 5,275,996, U.S. Pat. No. 5,641,722, U.S. Pat. No. 5,137,860, U.S. Pat. No. 5,095,125 or U.S. Pat. No. 4,933,312.

The catalysts of the invention can further comprise promoters. Suitable promoters are the elements of groups 1 to 15 of the Periodic Table and compounds thereof. Suitable promoters are, for example, described in the publications WO 97/12674 and WO 95/26817 and in the U.S. Pat. No. 5,137,860, U.S. Pat. No. 5,296,436, U.S. Pat. No. 5,158,923 and U.S. Pat. No. 4,795,818. Compounds of the elements cobalt, molybdenum, iron, zinc, hafnium, zirconium, lithium, titanium, chromium, manganese, nickel, copper, boron, silicon, antimony, tin, niobium and bismuth, particularly preferably molybdenum, iron, zinc, antimony, bismuth, lithium, are preferably used as promoters. The promoted catalysts of the invention can comprise one or more promoters. The total content of promoters in the finished catalyst is generally not more than about 5% by weight, in each case calculated as oxide. Preferred catalysts are those which do not comprise any promoters and those which comprise molybdenum or iron.

The main steps of the preferred catalyst production with formation of a precursor powder, shaping and subsequent calcination are as follows.

(a) Reaction of a pentavalent vanadium compound with an organic, reducing solvent in the presence of a phosphorus compound with heating. This step can optionally be carried out in the presence of a dispersed, pulverulent support material. Reaction without addition of support material is preferred.

(b) Isolation of the vanadium-, phosphorus-, oxygen-comprising catalyst precursor (“VPO precursor”), e.g. by filtration of evaporation.

(c) Drying of the VPO precursor and preferably initial preactivation by heating at a temperature of from 250 to 350° C. Pulverulent support material and/or a pore former can then optionally be added to the dried and preferably heated VPO precursor powder.

(d) Shaping by conversion into the structure according to the invention. Shaping is preferably carried out by tableting, preferably with prior mixing with a lubricant such as graphite.

(e) Preactivation of the shaped VPO precursor by heating in an atmosphere comprising oxygen (O₂), hydrogen oxide (H₂O) and/or inert gas.

The mechanical and catalytic properties of the catalyst can be influenced by appropriate combinations of temperatures, treatment times and gas atmospheres matched to the respective catalyst system.

As pentavalent vanadium compounds, it is possible to use oxides, acids and inorganic and organic salts which comprise pentavalent vanadium, or mixtures thereof. Preference is given to using vanadium pentoxide (V₂O₅), ammonium metavanadate (NH₄VO₃) and ammonium polyvanadate ((NH₄)₂V₆O₁₆), in particular vanadium pentoxide (V₂O₅). The pentavalent vanadium compounds present as solids are used in the form of a powder, preferably in a particle size range from 50 to 500 μm.

As phosphorus compounds, it is possible to use phosphorus compounds having a reducing action, for example phosphorous acid, and also pentavalent phosphorus compounds, for example phosphorus pentoxide (P₂O₅), orthophosphoric acid (H₃PO₄), pyrophosphoric acid (H₄P₂O₇), polyphosphoric acids of the general formula Hₙ₊₂PₙO₃ₙ₊₁ where n≧3 or mixtures thereof. Preference is given to using pentavalent phosphorus compounds. The content of the compounds and mixtures mentioned is usually reported in % by weight based on H₃PO₄. Preference is given to using from 80 to 110% strength H₃PO₄, particularly preferably from 95 to 110% strength H₃PO₄ and very particularly preferably from 100 to 105% strength H₃PO₄.

As solvent having a reducing action, preference is given to using a primary or secondary, acyclic or cyclic, unbranched or branched, saturated alcohol having from 3 to 6 carbon atoms or a mixture thereof. Preference is given to using a primary or secondary, unbranched or branched C₃-C₆-alkanol or cyclopentanol or cyclohexanol.

Suitable alcohols which may be mentioned are n-propanol (1-propanol), isopropanol (2-propanol), n-butanol (1-butanol), sec-butanol (2-butanol), isobutanol (2-methyl-1-propanol), 1-pentanol, 2-pentanol, 3-pentanol, 2-methyl-1-butanol, 3-methyl-1-butanol, 3-methyl-2-butanol, 2,2-dimethyl-1-propanol, 1-hexanol, 2-hexanol, 3-hexanol, 2-methyl-1-hexanol, 3-methyl-1-pentanol, 4-methyl-1-pentanol, 3-methyl-2-pentanol, 4-methyl-2-pentanol, 2,2-dimethyl-1-butanol, 2,3-dimethyl-1-butanol, 3,3-dimethyl-1-butanol, 3,3-dimethyl-2-butanol, cyclopentanol, cyclohexanol and mixtures thereof.

Very particular preference is given to n-propanol (1-propanol), n-butanol (1-butanol), isobutanol (2-methyl-1-propanol), 1-pentanol, 2-methyl-1-butanol, 3-methyl-1-butanol and cyclohexanol, in particular isobutanol.

The components can be combined in various ways, for example in a stirred vessel. The amount of the solvent having a reducing action should be above the amount which is stoichiometrically required for reduction of the vanadium from the oxidation state +5 to an oxidation state in the range from +3.5 to +4.5. In general, the amount of solvent having a reduction action to be added is at least such an amount that it is sufficient for slurrying the pentavalent vanadium compound so as to make intensive mixing with the phosphorus compound added possible.

The slurry is heated to convert the abovementioned compounds and form the catalyst precursor. The temperature range to be selected is dependent on various factors, in particular the reducing action and the boiling point of the components. In general, a temperature of from 50 to 200° C., preferably from 100 to 200° C., is set. The reaction at elevated temperature generally takes a number of hours.

Promoter compounds can be added at any point in time. Suitable promoter compounds are, for example, the acetates, acetylacetonates, oxalates, oxides or alkoxides of the abovementioned promoter metals, for instance cobalt acetate, cobalt(II) acetylacetonate, cobalt(II) chloride, molybdenum(VI) oxide, molybdenum(III) chloride, iron(III) acetylacetonate, iron(III) chloride, zinc(II) oxide, zinc(II) acetylacetonate, lithium chloride, lithium oxide, bismuth(III) chloride, bismuth(III) ethylhexanoate, nickel(II) ethylhexanoate, nickel(II) oxalate, zirconyl chloride, zirconium(IV) butoxide, silicon(IV) ethoxide, niobium(V) chloride and niobium(V) oxide.

After the abovementioned heat treatment is complete, the catalyst precursor formed is isolated, with a cooling phase and a storage or aging phase for the cooled reaction mixture optionally being able to be inserted before isolation. In the isolation step, the solid catalyst precursor is separated off from the liquid phase. Suitable methods are, for example, filtration, decantation or centrifugation. The catalyst precursor is preferably isolated by filtration.

The isolated catalyst precursor can be processed further with or without prior washing. The isolated catalyst precursor is preferably washed with a suitable solvent in order, for example, to remove residual agent having a reducing action (e.g. alcohol) or degradation products thereof which still adhere(s) to the catalyst precursor. Suitable solvents are, for example, alcohols (e.g. methanol, ethanol, 1-propanol, 2-propanol), aliphatic and/or aromatic hydrocarbons (e.g. pentane, hexane, petroleum spirit, benzene, toluene, xylenes), ketones (e.g. acetone, 2-butanone, 3-pentanone), ethers (e.g. 1,2-dimethoxyethane, tetrahydrofuran, 1,4-dioxane) or mixtures thereof. If the catalyst precursor is washed, preference is given to using 2-propanone and/or methanol and particularly preferably methanol.

After isolation of the catalyst precursor or after washing, the solid is generally dried.

Drying can be carried out under various conditions. In general, it is carried out under reduced pressure or atmospheric pressure. The drying temperature is generally from 30 to 250° C. Drying is preferably carried out at a pressure of from 1 to 30 kPa abs and a temperature of from 50 to 200° C. in an oxygen-comprising or oxygen-free gas atmosphere, for example air or nitrogen.

In a preferred embodiment of the shaping operation, the catalyst precursor powder is intensively mixed with from about 2 to 4% by weight of graphite and predensified. The predensified particles are tableted to give the shaped catalyst body. Preference is given to using a graphite having a specific surface area of from 0.5 to 5 m²/g and a particle diameter d₅₀ of from 40 to 200 μm, as described in WO 2008/087116.

In a further embodiment of shaping, the catalyst precursor powder is intensively mixed with a pore former and treated further and shaped as described above. In general, pore formers are compounds which comprise carbon, hydrogen, oxygen and/or nitrogen and can be mostly removed again by sublimation, decomposition and/or vaporization during the subsequent activation of the catalyst. The finished catalyst can comprise residues or decomposition products of the pore former. Suitable pore formers are, for example, fatty acids such as palmitic acid or stearic acid, dicarboxylic acids such as oxalic acid or malonic acid, cyclodextrins or polyethylene glycols. The use of malonic acid is preferred.

Shaping is preferably carried out by tableting. Tableting is a process of press agglomeration. Here, a pulverulent bulk material is introduced into a pressing tool having a die between two punches and compacted by uniaxial compression and shaped to give a solid compact. This operation is divided into four parts: metered introduction, compaction (elastic deformation), plastic deformation and ejection. Tableting is carried out, for example, on rotary presses or eccentric presses.

To form the internal holes, the upper punch and/or lower punch has projecting pins.

The shaped VPO precursor is preactivated (calcined) by heating in an atmosphere comprising oxygen (O₂), hydrogen oxide (H₂O) and/or inert gas in a temperature range from 250 to 600° C.

Suitable inert gases are, for example, nitrogen, carbon dioxide and noble gases.

The calcination can be carried out batchwise, for example in a shaft furnace, tray furnace, muffle furnace or oven, or continuously, for example in a rotary tube furnace, belt calcination furnace or rotary bulb furnace. It can comprise successive different sections in respect of the temperature, e.g. heating, holding of a constant temperature or cooling, and successive different sections in respect of the atmospheres, for example oxygen-comprising, water vapor-comprising, oxygen-free gas atmospheres. Suitable preactivation processes are described, for example, in the U.S. Pat. No. 5,137,860 and U.S. Pat. No. 4,933,312 and the publication WO 95/29006. Particular preference is given to continuous calcination in a belt calcination furnace having at least two, for example from two to ten, calcination zones which optionally have a different gas atmosphere and a different temperature. The mechanical and catalytic properties of the catalyst can be influenced and thus set in a targeted way by a suitable combination of temperatures, treatment times and gas atmospheres matched to the respective catalyst system.

Preference is given to a calcination in which the catalyst precursor is
(i) heated to a temperature of from 200 to 350° C. in an oxidizing atmosphere having an oxygen content of from 2 to 21% by volume in at least one calcination zone and maintained under these conditions until the desired average oxidation state of the vanadium has been attained; and
(ii) heated to a temperature of from 300 to 500° C. in a nonoxidizing atmosphere having an oxygen content of 0.5% by volume and a hydrogen oxide content of from 20 to 75% by volume in at least one further calcination zone and maintained under these conditions for 0.5 hour.

In step (i), the catalyst precursor is kept at a temperature of from 200 to 350° C. and preferably from 250 to 350° C. in an oxidizing atmosphere having a content of molecular oxygen of generally from 2 to 21% by volume and preferably from 5 to 21% by volume for a period of time which enables the desired average oxidation state of the vanadium to be established. In general, mixtures of oxygen, inert gases (e.g. nitrogen or argon), hydrogen oxide (water vapor) and/or air and also air are used in step (i). As far as the catalyst precursor conveyed through the calcination zone(s) is concerned, the temperature during the calcination step (i) can be kept constant or on average increase or decrease. Since step (i) is generally preceded by a heating phase, the temperature will in general firstly increase and then oscillate toward the desired final value. The calcination zone of step (i) is therefore generally preceded by at least one further calcination zone for heating up the catalyst precursor.

The period of time for which the heat treatment in step (i) is continued in the process of the invention should preferably be selected so that an average oxidation state of the vanadium of from +3.9 to +4.4, preferably from +4.0 to +4.3, is established.

Since determination of the average oxidation state of vanadium during calcination is extremely difficult for reasons of apparatus and time, the period of time required is advantageously determined experimentally in preliminary tests. In general, this is carried out using a series of measurements in which the samples are heat treated under defined conditions and are taken from the system after different times, cooled and analyzed to determine the average oxidation state of vanadium.

The time required in step (i) is generally dependent on the nature of the catalyst precursor, the temperature set and the gas atmosphere selected, in particular the oxygen content. In general, the time in step (i) extends to a period of over 0.5 hour and preferably over 1 hour. In general, a time of up to 4 hours, preferably up to 2 hours, is sufficient to set the desired average oxidation state. However, under some conditions (e.g. low range of the temperature interval and/or low content of molecular oxygen), a period of over 6 hours can be required.

In step (ii), the catalyst intermediate obtained is kept at a temperature of from 300 to 500° C. and preferably from 350 to ≦50° C. in a nonoxidizing atmosphere having a content of molecular oxygen of 0.5% by volume and of hydrogen oxide (water vapor) of from 20 to 75% by volume, preferably from 30 to 60% by volume, for a period of ≧0.5 hour, preferably from 2 to 10 hours and particularly preferably from 2 to 4 hours. The nonoxidizing atmosphere generally comprises, in addition to the hydrogen oxide mentioned, predominantly nitrogen and/or noble gases such as argon, but this does not constitute a restriction. Gases such as carbon dioxide are in principle also suitable. The nonoxidizing atmosphere preferably comprises 40% by volume of nitrogen. As far as the catalyst precursor conveyed through the calcination zone(s) is concerned, the temperature during the calcination step (ii) can be kept constant or on average increase or decrease. If step (ii) is carried out at a higher or lower temperature than step (i), there is generally a heating or cooling phase between the steps (i) and (ii), which is optionally implemented in a further calcination zone. To make improved separation from the oxygen-comprising atmosphere of step (i) possible, this further calcination zone between (i) and (ii) can, for example, be flushed with inert gas such as nitrogen. Step (ii) is preferably carried out at a temperature which is from 50 to 150° C. higher than that in step (i).

In general, the calcination comprises a further step (iii) which is to be carried out after step (ii) and in which the calcined catalyst precursor is cooled in an inert gas atmosphere to a temperature of ≦300° C., preferably from ≦200° C. and particularly preferably ≦150° C.

In the calcination according to the process of the invention, further steps are possible before, between and/or after steps (i) and (ii) or (i), (ii) and (iii). Without constituting a limitation, further steps which may be mentioned are, for example, changes in the temperature (heating, cooling), changes in the gas atmosphere (setting of a different gas atmosphere), further hold times, transfer of the catalyst intermediate into other apparatuses or interruption of the overall calcination operation.

Since the catalyst precursor is generally at a temperature of <100° C. before commencement of calcination, it usually has to be heated before step (i). Heating can be carried out using various gas atmospheres. Heating is preferably carried out in an oxidizing atmosphere as defined under step (i) or an inert gas atmosphere as defined under step (iii). The gas atmosphere can also be changed during the heating phase. Particular preference is given to heating up in the oxidizing atmosphere which is also employed in step (i).

The invention further provides a process for preparing maleic anhydride, wherein a hydrocarbon having at least four carbon atoms is brought into contact with a bed of shaped catalyst bodies according to the invention in the presence of an oxygen-comprising gas in at least one reaction tube. Shell-and-tube reactors are generally used as reactors. Suitable shell-and-tube reactors are described, for example, in EP-B 1 261 424.

Suitable hydrocarbons for use in the process of the invention are aliphatic and aromatic, saturated and unsaturated hydrocarbons having at least four carbon atoms, for example 1,3-butadiene, 1-butene, cis-2-butene, trans-2-butene, n-butane, C₄ mixture, 1,3-pentadiene, 1,4-pentadiene, 1-pentene, cis-2-pentene, trans-2-pentene, n-pentane, cyclopentadiene, dicyclopentadiene, cyclopentene, cyclopentane, C₅ mixture, hexenes, hexanes, cyclohexane and benzene. Preference is given to using propane, 1-butene, cis-2-butene, trans-2-butene, n-butane, benzene or mixtures thereof, in particular propane, n-butane or benzene. Particular preference is given to using n-butane, for example as pure n-butane or as a component in n-butane-comprising gases and liquids. The n-butane used can, for example, originate from natural gas, from steam crackers or FCC plants.

The hydrocarbon is generally introduced in a quantity-regulated manner, i.e. with a defined amount per unit time being continually set. The hydrocarbon can be metered in in liquid or gaseous form. It is preferably metered in liquid form with subsequent vaporization before entering the shell-and-tube unit.

Oxidants used are oxygen-comprising gases such as air, synthetic air, a gas enriched with oxygen or “pure” oxygen, e.g. oxygen originating from a fractionation of air. The oxygen-comprising gas is also added in a quantity-regulated manner.

The process of the invention is carried out at a temperature of from 250 to 500° C. The temperature mentioned is, regardless of the type of reactor, in each case the average temperature of the heat transfer medium. When n-butane is used as hydrocarbon starting material, the process of the invention is preferably carried out at a temperature of from 380 to 460° C. and particularly preferably from 380 to 440° C. When propane is used, the process of the invention is preferably carried out in the range from 250 to 350° C. When benzene is used, the process of the invention is preferably carried out in the range from 330 to 450° C.

The process of the invention is advantageously carried out isothermally with a temperature profile which increases over the length of the reactor or using a combination of a temperature which increases over the length of the reactor and an isothermal mode of operation.

The process of the invention is advantageously carried out at an oxygen partial pressure of from 0.6 bar to 50 bar, preferably from 2 bar to 50 bar, particularly preferably from 3 bar to 50 bar, in particular from 4 bar to 50 bar.

The hydrocarbon concentration of the feed stream fed to the reactor unit is from 0.5 to 10% by volume, preferably from 0.8 to 10% by volume, particularly preferably from 1 to 10% by volume and very particularly preferably from 2 to 10% by volume.

The hydrocarbon conversion per pass through the reactor is from 40 to 100%, preferably from 50 to 95%, particularly preferably from 70 to 95% and in particular from 85 to 95%, of the hydrocarbon in the feed stream.

In the process of the invention, a GHSV (gas hourly space velocity) of preferably from 1000 to 10 000 h⁻¹ and particularly preferably from 1500 to 4000 h⁻¹, based on the volume of the feed stream standardized to 0° C. and 0.1013 MPa abs and based on the reaction volume which is filled with catalyst or whose geometric surface area is coated with catalyst, is preferably set via the amount of feed stream introduced into the reactor unit.

The process of the invention can be carried out in two preferred process variants, viz. the variant in a “single pass” and the variant with “recirculation”. In a “single pass”, maleic anhydride and optionally oxygenated hydrocarbon by-products are removed from the reactor discharge and the remaining gas mixture discharged from the process and optionally utilized thermally. In the case of “recirculation” maleic anhydride and optionally oxygenated hydrocarbon by-products are likewise removed from the reactor discharge and the remaining gas mixture, which comprises unreacted hydrocarbon, is recirculated in its entirety or in part to the reactor. A further variant of “recirculation” is removal of the unreacted hydrocarbon and recirculation thereof to the reactor.

The reaction products or the product stream can optionally be diluted by addition of materials which are inert under the reaction conditions, for example water or nitrogen, either at the end of the reactor or at the reactor outlet so as to give a nonexplosive product stream. Furthermore, a nonexplosive product stream can advantageously be achieved by means of a pressure stage. This product stream can then be worked up by means of conventional work-up units.

When n-butane is used, a volatile phosphorus compound is advantageously introduced into the gas in the process of the invention to ensure a long catalyst operating life and a further increase in the conversion, selectivity, yield, space velocity of the catalyst and space-time yield. Its concentration at the beginning, i.e. at the reactor inlet, is from 0.2 to 20 ppm by volume of the volatile phosphorus compounds based on the total volume of the gas at the reactor inlet. Preference is given to a content of from 0.5 to 5% by volume. Volatile phosphorus compounds are all phosphorus-comprising compounds which are present in gaseous form in the desired concentration under the use conditions. Preference is given to using triethyl phosphate or trimethyl phosphate as volatile phosphorus compound.

The invention is illustrated by the following examples.

### DEFINITIONS

The parameters used in this text are, unless indicated otherwise, defined as follows:

Bulk density ρ=Bulk density of the shaped catalyst body [in kg/l]

geometric surface area A_{geo}=geometric surface area of the shaped bodies [mm²]

geometric volume V_{geo}=geometric volume of the shaped bodies [mm³]

x_{n-butane}=butane concentration of the feed stream

X_{n-butane}=n-butane conversion

x_{TEP}=triethyl phosphate concentration of the feed stream

x_{H2O}=water vapor concentration of the feed stream

GHSV=quantity of the feed stream, based on the volume standardized to 0° C. and 0.1013 MPa abs of the feed stream introduced and based on the reaction volume which is filled with catalyst

### Determination of the Bulk Density in a Model Tube

To determine the bulk density, shaped catalyst bodies are freed of dust and fragments by a gentle sieving motion on a sieve having a mesh opening of 5 mm. These shaped bodies are introduced via a vibratory chute into a reaction tube having a length of 650 cm and an internal diameter of 21 mm for a period of from 180 to 200 s until the tube is filled to a fill height of 600 cm±1 cm. Care is taken to ensure that the tube is uniformly filled and the filling time is in the defined range. When the fill height (600 cm±1 cm) is reached, the mass of the catalyst introduced is determined. The bulk density=m (catalyst) in kg/V(reactor) in I, where V(reactor) is the product of fill height and tube cross section.

### Determination of the Residual Isobutanol Content in the Dried Catalyst Precursor

To determine the residual isobutanol content, about 4 g of the dried pulverulent catalyst precursor and about 10 g of N,N-dimethylformamide were weighed accurately into a heatable stirred apparatus provided with reflux condenser. The mixture was subsequently heated to the boiling point while stirring and maintained under these conditions for 30 minutes. After cooling, the suspension was filtered and the isobutanol content of the filtrate was determined quantitatively by gas chromatography. The residual isobutanol content was then calculated from the average concentration of isobutanol in the N,N-dimethylformamide and the weighed out amounts of N,N-dimethylformamide and catalyst precursor.

### Determination of the Lateral Compressive Strength of the Hollow Cylinders

To determine the lateral compressive strength, the shaped catalyst bodies were placed with the rounded side surface on in each case the flat metal support plate of an appropriate measurement device in successive measurements. The two parallel flat end faces were thus oriented vertically. A flat metal punch was then driven down from the top onto the shaped catalyst body at an advanced rate of 1.6 mm/min and the force applied to the shaped catalyst body was recorded as a function of time until fracture of the body occurred. The lateral compressive strength of the individual shaped catalyst body corresponds to the maximum force applied.

### Determination of the Pore Volume

The specific pore volume was determined by mercury porosimetry in accordance with DIN 66133.

### Production of the Catalyst Precursor

6.1 m³ of isobutanol were placed in a stirred 8 m³ steel/enamel vessel which was blanketed with nitrogen, could be heated externally by means of pressurized water and was provided with baffles. After the three-stage impeller stirrer was started, the isobutanol was heated to 90° C. under reflux. At this temperature, the addition of 736 kg of vanadium pentoxide via the transport screw was commenced. After about ⅔ of the desired amount of vanadium pentoxide had been added after about 20 minutes, the introduction of 900 kg of 105% strength phosphoric acid by pumping was commenced while continuing to add vanadium pentoxide. To clear the pump, a further 0.2 m³ of isobutanol were pumped in afterward. The reaction mixture was subsequently heated to about 100-108° C. under reflux and maintained under these conditions for 14 hours. The hot suspension was subsequently drained into a pressure filter which was blanketed with nitrogen and heated and the solid was filtered off at a temperature of about 100° C. at a pressure above the filter of up to 0.35 MPa abs. The filtercake was blow dried over a period of about one hour by continual introduction of nitrogen at 100° C. while stirring by means of a centrally arranged stirrer whose height could be adjusted. After blowing dry, the solid was heated to about 155° C. and evacuated to a pressure of 15 kPa abs (150 mbar abs). Drying was carried out to a residual isobutanol content of <2% by weight in the dried catalyst precursor.

The dried powder obtained was then heated for 2 hours in air in a rotary tube having a length of 6.5 m, an internal diameter of 0.9 m and internal helices. The speed of rotation of the rotary tube was 0.4 rpm. The powder was conveyed into the rotary tube in an amount of 60 kg/h. The inflow of air was 100 m³/h. The temperatures of the five heating zones of equal length measured directly on the outside of the rotary tube were 250° C., 300° C., 340° C., 340° C. and 340° C. After cooling to room temperature, the catalyst precursor was intimately mixed with 1% by weight of graphite and compacted in a roller compactor. The fines having a particle size of <400 μm in the compacted material were sieved off and fed back to compacting. The coarse material having a particle size of 400 μm was intimately mixed with a further 2% by weight of graphite. This will hereinafter be referred to as “catalyst precursor powder”.

### Production of the Catalysts 1 to 9

To produce the catalysts, the catalyst precursor powder was mixed with the amount indicated in the table of malonic acid as pore former. The catalyst precursor powder or the mixture with malonic acid was tableted in a tableting machine to give hollow cylinders or cylinders having four through-holes, with the dimensions indicated in the table (external diameter d₁*height h*diameter of the hole(s) d₂).

The tableted catalyst precursor specimens were subsequently introduced into a belt calciner and calcined as follows, with the residence time in each individual zone being about 1.78 h.

Preactivation parameters for the catalysts (with malonic acid)

| Zone | Temperature | Fresh gas introduced |
|---|---|---|
| Calcination zone 1 | 150° C. | Air |
| Calcination zone 2 | 180° C. | Air |
| Calcination zone 3 | 280° C. | Air, N₂/H₂O vapor |
| | | (5% volume of O₂) |
| Calcination zone 4 | 325° C. | Air, N₂/H₂O vapor |
| | | (5% volume of O₂) |
| Transition zone | Cooling to | |
| | 200° C. | |
| Calcination zone 5 | 335° C. | N₂ |
| Calcination zone 6 | 400° C. | N₂/H₂O vapor (1:1) |
| Calcination zone 7 | 425° C. | N₂/H₂O vapor (1:1) |
| Calcination zone 8 | 355° C. | N₂ |

Preactivation parameters for catalyst (without pore former)

| Zone | Temperature | Fresh gas introduced |
|---|---|---|
| Calcination zone 1 | 140° C. | Air |
| Calcination zone 2 | 140° C. | Air |
| Calcination zone 3 | 260° C. | Air |
| Calcination zone 4 | 300° C. | Air |
| Transition zone | Cooling to | Air |
| | 200° C. | |
| Calcination zone 5 | 335° C. | N₂ |
| Calcination zone 6 | 400° C. | N₂/H₂O vapor (1:1) |
| Calcination zone 7 | 425° C. | N₂/H₂O vapor (1:1) |
| Calcination zone 8 | 355° C. | N₂ |

### Catalytic Tests

The test plant was equipped with a feed unit and a reactor tube. Replacement of a shell-and-tube reactor by a reactor tube is very readily possible on the laboratory or pilot plant scale as long as the dimensions of the reactor tube are in the region of those of an industrial reactor tube. The plant was operated in a “single pass”.

The hydrocarbon was introduced in liquid form in a quantity-regulated manner by means of a pump. As oxygen-comprising gas, air was introduced in a quantity-regulated manner. Triethyl phosphate (TEP) was likewise introduced in a quantity-regulated manner, in liquid form dissolved in water.

The shell-and-tube reactor unit comprised a shell-and-tube reactor having one reactor tube. The length of the reactor tube was 6.5 m, and the internal diameter was 22.3 mm. A multi-thermocouple having 20 temperature measuring points was located in a protective tube having an external diameter of 6 mm within the reactor tube. Heating of the reactor was effected by means of a heat transfer medium circuit having a length of 6.5 m. A salt melt was used as heat transfer medium. The reaction gas mixture flowed from the top downward through the reactor tube. The upper 0.2 m of the 6.5 m long reactor tube remained unfilled. This was followed by a 0.3 m long preheating zone filled with shaped steatite bodies as inert material. The preheating zone was followed by the catalyst bed which comprised a total of 2173 ml of catalyst.

Gaseous product was taken off immediately downstream of the shell-and-tube reactor unit and passed to gas-chromatographic on-line analysis. The main stream of the gaseous reactor discharge was discharged from the plant.

The reaction conditions for catalytic testing were as follows: x_{n-butane}=2% by volume, GHSV=2000 h⁻¹, Pᵢₙ=2.3 barg, X_{n-butane}=85%, x_{TEP}=2.25-2.5 ppm by volume, x_{H2O}=3% by volume.

The measurements were carried out after a minimum running time of the catalyst of 150 h.

TABLE
Geometric characterization and catalyst properties

| Catalysts | 1 | 2 | 3 | 4 | 5 | 6 | 7* | 8 | 9 |
|---|---|---|---|---|---|---|---|---|---|
| d₁ * h * d₂ | 6 * 4.2 * 3.5 | 6 * 4.2 * 3.5 | 6 * 4.2 * 3.5 | 6 * 4.2 * 3 | 6 * 4.2 * 3 | 5 * 3.2 * 2.5 | 6.5 * 4.2 * 3.7 | 6.5 * 4.2 * 1.85 | 6.5 * 4.2 * 1.85 |
| [mm] | | | | | | | | | |
| Number of | 1 | 1 | 1 | 1 | 1 | 1 | 1 | 4 | 4 |
| holes | | | | | | | | | |
| Pore former | 10 | 20 | 25 | 15 | 30 | 0 | 20 | 0 | 20 |
| [% by | | | | | | | | | |
| weight] | | | | | | | | | |
| A_{geo}/V_{geo} | 1.84 | 1.84 | 1.84 | 1.57 | 1.57 | 1.91 | 1.67 | 2.18 | 2.18 |
| [mm⁻¹] | | | | | | | | | |
| PV [ml/g] | 0.33 | 0.36 | 0.42 | 0.36 | 0.49 | 0.27 | 0.368 | 0.336 | 0.408 |
| ρ [kg/l] | 0.55 | 0.51 | 0.47 | 0.58 | 0.49 | 0.65 | 0.442 | 0.458 | 0.417 |
| PV * ρ * | 0.334 | 0.337 | 0.363 | 0.33 | 0.38 | 0.335 | 0.272 | 0.336 | 0.371 |
| A_{geo}/V_{geo} | | | | | | | | | |
| MAn yield | 57.1 | 56.8 | 58.2 | 56.8 | 59.2 | 57 | 54.5 | 57.9 | 58.9 |
| [mol %] | | | | | | | | | |

*Comparative example

The results show that the MAn yield correlates with the product PV·ρ·A_{geo}/V_{geo}. Appropriate selection of the pore volume PV and the shaped body geometry (A_{geo}/V_{geo}) makes it possible to produce shaped catalyst bodies which combine a low pressure drop with a high MAn yield.

1.-11. (canceled)

12. A shaped catalyst body which has an essentially cylindrical body having a longitudinal axis, where the cylindrical body has at least one internal hole running right through the body essentially parallel to the cylindrical axis of the body, and whose catalytically active composition comprises a vanadium- and phosphorus-comprising multielement oxide, wherein the specific pore volume PV (in ml/g) of the shaped catalyst body, the bulk density ρ of the shaped catalyst body (in kg/l), the geometric surface area A_{geo} (in mm²) and the geometric volume V_{geo} (in mm³) of the shaped catalyst body fulfill the condition:
0.275<PV·ρ·A_{geo}/V_{geo},
the bulk density ρ is less than 0.60 kg/l and the specific pore volume PV is at least 0.30 ml/g.

13. The shaped catalyst body according to claim 12, wherein A_{geo}/V_{geo} is at least 1.50 mm⁻¹.

14. The shaped catalyst body according to claim 12, wherein at least 15% of the specific pore volume is formed by pores having a size of from 0.3 to 20 μm.

15. A process for preparing maleic anhydride, wherein a hydrocarbon having at least four carbon atoms is brought into contact with a bed of shaped catalyst bodies according to claim 12 in the presence of an oxygen-comprising gas in at least one reaction tube.

16. A process for producing a shaped catalyst body according to claim 12, wherein a vanadium- and phosphorus-comprising multielement oxide or a precursor thereof is mixed with a pore former, the mixture is shaped to give cylindrical shaped bodies having a longitudinal axis and at least one hole which runs right through the body essentially parallel to the cylindrical axis of the body, where A_{geo}/V_{geo} is at least 1.50 mm⁻¹, and the shaped bodies are calcined.

17. The process according to claim 16, wherein from 18 to 40% by weight of pore former, based on the weight of the multielement oxide or the precursor thereof, is used.

18. The process according to claim 16, wherein the pore former is particulate and has a particle size distribution having an average particle diameter d₅₀ in the range from 1 to 80 μm.

19. The process according to claim 16, wherein the pore former is malonic acid.
